(19) United States
(12) Patent Application Publication
(10) Pub. No.: US 2025/0341041 A1
(43) Pub. Date: Nov. 6, 2025

(54) LAUNDRY APPLIANCE AND METHODS OF OZONE DISPENSING

(71) Applicant: Haier US Appliance Solutions, Inc., Wilmington, DE (US)

(72) Inventors: Nemetalla Fahed Salameh, Louisville, KY (US); Alexander B. Leibman, Prospect, KY (US)

(21) Appl. No.: 18/652,053

(22) Filed: May 1, 2024

(51) Int. Cl. D06F 58/20; A61L 2/20; D06F 34/14; D06F 58/02; D06F 58/44; D06F 103/60; D06F 105/20; D06F 105/26; D06F 105/38

(52) U.S. Cl. CPC D06F 58/203; A61L 2/202; D06F 34/14; D06F 58/02; D06F 58/206; D06F 58/44; A61L 2202/14; A61L 2202/17; A61L 2202/26; D06F 2103/60; D06F 2105/20; D06F 2105/26; D06F 2105/38

(57) ABSTRACT

A laundry appliance may include a cabinet, a tub, a laundry basket, a conditioning system, and a controller. The tub may be positioned within the cabinet. The laundry basket may define a laundry chamber for receipt of articles for washing or drying. The conditioning system may include a heat exchanger disposed within the cabinet in thermal communication with the laundry chamber to adjust a temperature therein. The controller may be configured to initiate a laundry operation. The laundry operation may include initiating a cooling action at the heat exchanger to reduce a temperature within the laundry chamber, directing ozone transmission into the laundry chamber following initiating the cooling action, initiating a heating action at the laundry chamber to increase the temperature within the laundry chamber following directing ozone transmission, and determining achievement of a set ozone depletion level within the laundry chamber following initiating the heating action.

### FIELD OF THE DISCLOSURE

The present subject matter relates generally to laundry appliances, such dryer or combined washer/dryer appliances, and methods for operating the same.

### BACKGROUND OF THE DISCLOSURE

Laundry appliance, such as dryers or drying appliances, generally include a cabinet with a drum mounted therein. For dryers, a heater or heater assembly is also often provided to pass heated air through the chamber of the drum in order to dry moisture-laden articles disposed within the chamber. This may be provided in the context of a dedicated drying appliance or a combination washing and drying appliance, which may greatly increase the ease and convenience for cleaning clothing articles.

Operating cycles in conventional laundry appliances are initiated by a user of the appliance. For instance, the user must select the desired cycle type (e.g., cotton, mixed, etc.) and operating parameters before manually starting the wash or drying cycle. Although conventional laundry appliances are configured to use typical methods for cleaning laundry (e.g., water, detergent, wash additives, etc.), such methods have drawbacks. For instance, such methods can be harsh on fabrics or insufficient to eliminate certain microbes (e.g., bacteria or fungi).

In order to address such concerns, attempts have been made to include an ozone generator for producing gaseous ozone (i.e., O₃) within a laundry appliance, which may be capable of reducing or eliminating various microbes. However, these existing systems suffer a number of drawbacks. Specifically, having sufficient ozone for sanitization can be difficult to achieve in a laundry appliance. Moreover, large concentrations ozone [e.g., ozone in concentrations above 50 parts per billion (ppb)] may cause respiratory irritation or have other deleterious effects. Ozone generated may eventually break down as oxygen (i.e., O₂), but the process can be relatively slow.

As a result, it would be useful to provide an appliance or method of operation addressing one or more of the above concerns.

### BRIEF DESCRIPTION OF THE DISCLOSURE

Aspects and advantages of the invention will be set forth in part in the following description, or may be obvious from the description, or may be learned through practice of the invention.

In one exemplary aspect of the present disclosure, a laundry appliance is provided. The laundry appliance may include a cabinet, a tub, a laundry basket, a conditioning system, and a controller. The tub may be positioned within the cabinet. The tub may define a tub outlet and a tub inlet. The laundry basket may be rotatably mounted within the tub. The laundry basket may define a laundry chamber for receipt of articles for washing or drying. The conditioning system may include a heat exchanger disposed within the cabinet in thermal communication with the laundry chamber to adjust a temperature therein. The controller may be operably coupled to the conditioning system and configured to initiate a laundry operation. The laundry operation may include initiating a cooling action at the heat exchanger to reduce a temperature within the laundry chamber, directing ozone transmission into the laundry chamber following initiating the cooling action, initiating a heating action at the laundry chamber to increase the temperature within the laundry chamber following directing ozone transmission, and determining achievement of a set ozone depletion level within the laundry chamber following initiating the heating action.

In another exemplary aspect of the present disclosure, a method of operating a laundry appliance is provided. The method may include initiating a cooling action at a heat exchanger to reduce a temperature within a laundry chamber. The method may also include directing ozone transmission into the laundry chamber following initiating the cooling action. The method may further include initiating a heating action at the laundry chamber to increase the temperature within the laundry chamber following directing ozone transmission. The method may still further include determining achievement of a set ozone depletion level within the laundry chamber following initiating the heating action.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures.

FIG. 1 provides a perspective view of a laundry appliance in accordance with exemplary embodiments of the present disclosure.

FIG. 2 provides a side sectional view of the exemplary laundry appliance of FIG. 1.

FIG. 3 provides a schematic diagram of an exemplary heat pump dryer appliance and a conditioning system thereof in accordance with exemplary embodiments of the present disclosure.

FIG. 4 illustrates a method for operating a laundry appliance in accordance with exemplary embodiments of the present disclosure.

Repeat use of reference characters in the present specification and drawings is intended to represent the same or analogous features or elements of the present invention.

### DETAILED DESCRIPTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope of the invention. For instance, features illustrated or described as part of one embodiment can be used with another embodiment to yield a still further embodiment. Thus, it is intended that the present invention covers such modifications and variations as come within the scope of the appended claims and their equivalents. The word “exemplary” is used herein to mean “serving as an example, instance, or illustration.” In addition, references to “an embodiment” or “one embodiment” does not necessarily refer to the same embodiment, although it may. Any implementation described herein as “exemplary” or “an embodiment” is not necessarily to be construed as preferred or advantageous over other implementations.

As used herein, the terms “first,” “second,” and “third” may be used interchangeably to distinguish one component from another and are not intended to signify location or importance of the individual components. The terms “includes” and “including” are intended to be inclusive in a manner similar to the term “comprising.” Similarly, the term “or” is generally intended to be inclusive (i.e., “A or B” is intended to mean “A or B or both”). In addition, here and throughout the specification and claims, range limitations may be combined or interchanged. Such ranges are identified and include all the sub-ranges contained therein unless context or language indicates otherwise. For example, all ranges disclosed herein are inclusive of the endpoints, and the endpoints are independently combinable with each other. The singular forms “a,” “an,” and “the” include plural references unless the context clearly dictates otherwise.

Approximating language, as used herein throughout the specification and claims, may be applied to modify any quantitative representation that could permissibly vary without resulting in a change in the basic function to which it is related. Accordingly, a value modified by a term or terms, such as “generally,” “about,” “approximately,” and “substantially,” are not to be limited to the precise value specified. In at least some instances, the approximating language may correspond to the precision of an instrument for measuring the value, or the precision of the methods or machines for constructing or manufacturing the components or systems. For example, the approximating language may refer to being within a 10 percent margin (i.e., including values within ten percent greater or less than the stated value). In this regard, for example, when used in the context of an angle or direction, such terms include within ten degrees greater or less than the stated angle or direction (e.g., “generally vertical” includes forming an angle of up to ten degrees in any direction, such as, clockwise or counterclockwise, with the vertical direction V).

The terms “upstream” and “downstream” refer to the relative flow direction with respect to fluid flow in a fluid pathway. For example, “upstream” refers to the flow direction from which the fluid flows, and “downstream” refers to the flow direction to which the fluid flows.

Except as explicitly indicated otherwise, recitation of a singular processing element (e.g., “a controller,” “a processor,” “a microprocessor,” etc.) is understood to include more than one processing element. In other words, “a processing element” is generally understood as “one or more processing element.” Furthermore, barring a specific statement to the contrary, any steps or functions recited as being performed by “the processing element” or “said processing element” are generally understood to be capable of being performed by “any one of the one or more processing elements.” Thus, a first step or function performed by “the processing element” may be performed by “any one of the one or more processing elements,” and a second step or function performed by “the processing element” may be performed by “any one of the one or more processing elements and not necessarily by the same one of the one or more processing elements by which the first step or function is performed.” Moreover, it is understood that recitation of “the processing element” or “said processing element” performing a plurality of steps or functions does not require that at least one discrete processing element be capable of performing each one of the plurality of steps or functions.

Aspects of the present disclosure may provide a laundry appliance advantageously capable of sanitizing articles (e.g., without using water, detergent, wash additives, etc. for sanitization), such as by generating ozone, while preventing undesirable exposure for users. For instance, the laundry appliance may notably include features or methods for cooling a laundry (e.g., wash or dry) chamber before ozone is dispensed, then heating the chamber to quickly reduce or eliminate the ozone before the user opens the appliance.

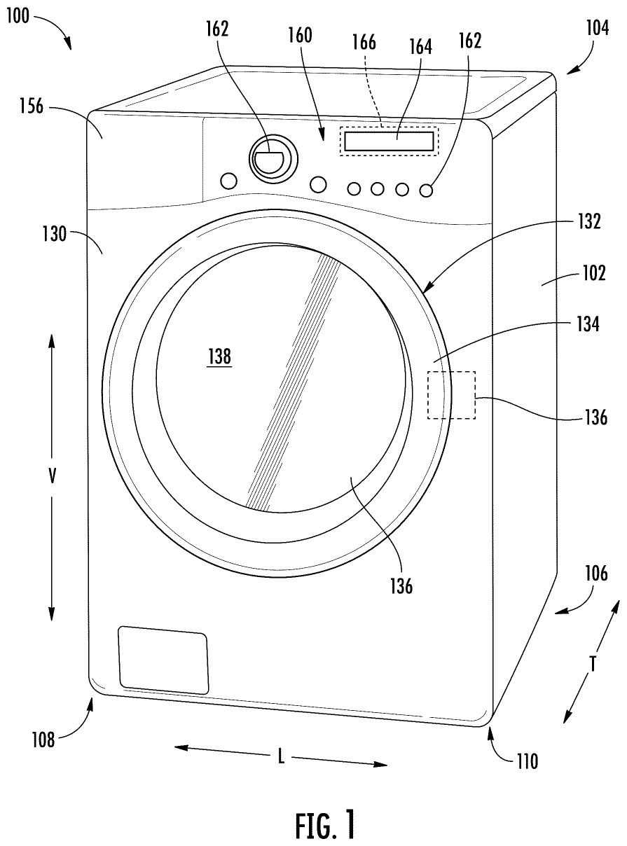

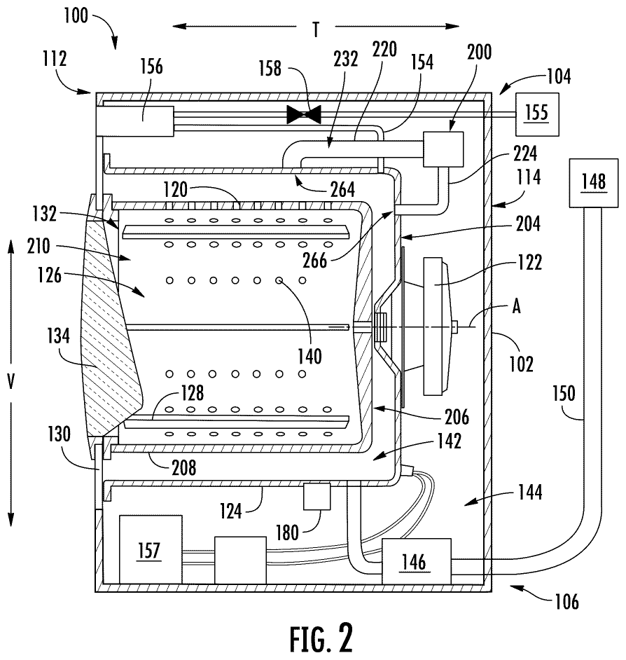

Referring now to the figures, an exemplary laundry appliance that may be used to implement aspects of the present disclosure will be described. Specifically, FIG. 1 is a perspective view of an exemplary horizontal axis washer/dryer appliance 100 (e.g., washer and condenser dryer combination appliance), referred to herein for simplicity as laundry appliance 100. FIG. 2 is a side sectional view of laundry appliance 100. As illustrated, laundry appliance 100 generally defines a vertical direction V, a lateral direction L, and a transverse direction T, each of which is mutually perpendicular, such that an orthogonal coordinate system is generally defined. Laundry appliance 100 includes a cabinet 102 that extends between a top 104 and a bottom 106 along the vertical direction V, between a left side 108 and a right side 110 along the lateral direction, and between a front 112 and a rear 114 along the transverse direction T.

Referring to FIG. 2, a laundry basket 120 is rotatably mounted within cabinet 102 such that it is rotatable about an axis of rotation A. According to the illustrated embodiment, axis of rotation A is substantially parallel to a horizontal direction (e.g., the transverse direction T), as this exemplary appliance is a front load appliance. A motor 122, such as a pancake motor, is in mechanical communication with laundry basket 120 to selectively rotate laundry basket 120 (e.g., during an agitation or a rinse phase of laundry appliance 100). Motor 122 may be mechanically coupled to laundry basket 120 directly or indirectly (e.g., via a pulley and a belt—not pictured). Laundry basket 120 is received within a tub 124 that defines a (e.g., laundry or drying) chamber 126 that is configured for receipt of articles for washing or drying.

As used herein, the terms “clothing” or “articles” includes but need not be limited to fabrics, textiles, garments, linens, papers, or other items from which the extraction of moisture is desirable. Furthermore, the term “load” or “laundry load” refers to the combination of clothing that may be washed together or dried together in laundry appliance 100 (e.g., the combination washer and dryer) and may include a mixture of different or similar articles of clothing of different or similar types and kinds of fabrics, textiles, garments and linens within a particular laundering process.

The tub 124 holds wash and rinse fluids for agitation in laundry basket 120 within tub 124. As used herein, “wash fluid” may refer to water, detergent, fabric softener, bleach, or any other suitable wash additive or combination thereof. Indeed, for simplicity of discussion, these terms may all be used interchangeably herein without limiting the present subject matter to any particular “wash fluid.”

Laundry basket 120 may define one or more agitator features that extend into chamber 126 to assist in agitation, cleaning, and drying of articles disposed within chamber 126 during operation of laundry appliance 100. For example, as illustrated in FIG. 2, a plurality of baffles or ribs 128 extend from basket 120 into chamber 126. In this manner, for example, ribs 128 may lift articles disposed in laundry basket 120 and then allow such articles to tumble back to a bottom of drum laundry basket 120 as it rotates. Ribs 128 may be mounted to laundry basket 120 such that ribs 128 rotate with laundry basket 120 during operation of laundry appliance 100.

Referring generally to FIGS. 1 and 2, cabinet 102 may include a front panel 130 which defines an opening 132 that permits user access to laundry basket 120 and tub 124. More specifically, laundry appliance 100 includes a door 134 that is positioned over opening 132 and is rotatably mounted to front panel 130. In this manner, door 134 permits selective access to opening 132 by being movable between an open position facilitating access to a tub 124 and a closed position (FIG. 1) prohibiting access to tub 124. Laundry appliance 100 may further a latch assembly 136 (see FIG. 1), such as a separable moving latch (e.g., mounted to cabinet 102) and catch (e.g., fixed to door 134), for selectively locking door 134 in the closed position or detecting the door 134 in the closed position. In particular, latch assembly 136 may be configured to place the door 134 (and thus chamber 126) in a locked state or an unlocked state (e.g., based on whether latch assembly 136 engages door 134 to be held shut, as is understood). Latch assembly 136 may be desirable, for example, to ensure only secured access to chamber 126 or to otherwise ensure and verify that door 134 is closed during certain operating cycles or events.

In some embodiments, a window 138 in door 134 permits viewing of laundry basket 120 when door 134 is in the closed position (e.g., during operation of laundry appliance 100). Door 134 may include a handle (not shown) that, for example, a user may pull when opening and closing door 134. Further, although door 134 is illustrated as mounted to front panel 130, it should be appreciated that door 134 may be mounted to another side of cabinet 102 or any other suitable support according to alternative embodiments.

Referring again to FIG. 2, laundry basket 120 may also define a plurality of perforations 140 in order to facilitate fluid communication between an interior of basket 120 and tub 124. A sump 142 is defined by tub 124 at a bottom of tub 124 along the vertical direction V. Thus, sump 142 is configured for receipt of and generally collects wash fluid during operation of laundry appliance 100. For example, during operation of laundry appliance 100, wash fluid may be urged by gravity from basket 120 to sump 142 through plurality of perforations 140.

In some embodiments, a drain pump assembly 144 is located beneath tub 124 and is in fluid communication with sump 142 for periodically discharging soiled wash fluid from laundry appliance 100. Drain pump assembly 144 may generally include a drain pump 146 which is in fluid communication with sump 142 and with an external drain 148 through a drain hose 150. During a drain cycle or phase (e.g., as a portion of a wash cycle), drain pump 146 urges a flow of wash fluid from sump 142, through drain hose 150, and to external drain 148. More specifically, drain pump 146 includes a motor (not shown) which is energized during a drain cycle such that drain pump 146 draws wash fluid from sump 142 and urges it through drain hose 150 to external drain 148.

A spout 154 is configured for directing a flow of fluid into tub 124. For example, spout 154 may be in fluid communication with a water supply 155 (FIG. 2) in order to direct fluid (e.g., clean water or wash fluid) into tub 124. Spout 154 may also be in fluid communication with the sump 142. For example, pump assembly 144 may direct wash fluid disposed in sump 142 to spout 154 in order to circulate wash fluid in tub 124.

As illustrated in FIG. 2, a detergent drawer 156 may be slidably mounted within front panel 130. Detergent drawer 156 receives a wash additive (e.g., detergent, fabric softener, bleach, or any other suitable liquid or powder) and directs the fluid additive to wash chamber 126 during operation of laundry appliance 100. According to the illustrated embodiment, detergent drawer 156 may also be fluidly coupled to spout 154 to facilitate the complete and accurate dispensing of wash additive.

In optional embodiments, a bulk reservoir 157 is disposed within cabinet 102 and is configured for receipt of fluid additive or detergent for use during operation of laundry appliance 100. Moreover, bulk reservoir 157 may be sized such that a volume of fluid additive sufficient for a plurality or multitude of wash cycles of laundry appliance 100 (e.g., five, ten, twenty, fifty, or any other suitable number of wash cycles) may fill bulk reservoir 157. Thus, for example, a user can fill bulk reservoir 157 with fluid additive and operate laundry appliance 100 for a plurality of wash cycles without refilling bulk reservoir 157 with fluid additive. A reservoir pump may be configured for selective delivery of the fluid additive from bulk reservoir 157 to tub 124.

A water supply valve or control valve 158 may provide a flow of water from a water supply source (such as a municipal water supply 155) into detergent dispenser 156 or into tub 124. In this manner, control valve 158 may generally be operable to supply water into detergent dispenser 156 to generate a wash fluid (e.g., for use in a wash cycle) or a flow of fresh water (e.g., for a rinse phase). It should be appreciated that control valve 158 may be positioned at any other suitable location within cabinet 102.

A control panel 160 including a plurality of input selectors 162 (e.g., buttons, knobs, toggles, touch screens, etc.) is coupled to front panel 130. Control panel 160 and input selectors 162 collectively form a user interface input for operator selection of machine cycles and features. For example, in one embodiment, a display 164 indicates selected features, a countdown timer, or other items of interest to machine users.

Operation of laundry appliance 100 is controlled by a controller or processing device 166 (FIG. 1) that is operatively coupled to control panel 160 for user manipulation to select laundry cycles and features. In response to user manipulation of control panel 160, controller 166 operates the various components of laundry appliance 100 to execute selected machine cycles and features.

Controller 166 may include a memory and microprocessor, such as a general or special purpose microprocessor operable to execute programming instructions or micro-control code associated with a cleaning cycle. The memory may represent random access memory such as DRAM, or read only memory such as ROM or FLASH. In one embodiment, the processor executes programming instructions stored in memory. The memory may be a separate component from the processor or may be included onboard within the processor. Alternatively, controller 166 may be constructed without using a microprocessor (e.g., using a combination of discrete analog or digital logic circuitry-such as switches, amplifiers, integrators, comparators, flip-flops, AND gates, and the like) to perform control functionality instead of relying upon software. Control panel 160 and other components of laundry appliance 100 may be in communication with controller 166 via one or more signal lines or shared communication busses.

During operation of laundry appliance 100, laundry items are loaded into laundry basket 120 through opening 132, and a laundry operation; such as a washing, wash/dry (e.g., having discrete wash and dry cycles), or drying operation; is initiated through operator manipulation of input selectors 162. In a washing or wash/dry operation including a wash cycle (e.g., including a rinse cycle), tub 124 is filled with water, detergent, or other fluid additives (e.g., via spout 154 and or detergent drawer 156). One or more valves (e.g., control valve 158) can be controlled by laundry appliance 100 to provide for filling laundry basket 120 to the appropriate level for the amount of articles being washed or rinsed. By way of example for a wash cycle, once laundry basket 120 is properly filled with fluid, the contents of laundry basket 120 can be agitated (e.g., with ribs 128) for washing of articles in laundry basket 120.

After an agitation phase of the wash cycle is completed, tub 124 can be drained. Laundry articles can then be rinsed by again adding fluid to tub 124, depending on the particulars of the cleaning cycle selected by a user. Ribs 128 may again provide agitation within laundry basket 120. One or more spin cycles or phases may also be used. In particular, a spin phase may be applied after the wash cycle or after the rinse phase in order to wring wash fluid from the articles being washed. During a final spin cycle, basket 120 is rotated at relatively high speeds and drain pump assembly 144 may discharge wash fluid from sump 142. Following the wash cycle, a dry cycle may be executed, as will be described in greater detail below.

While described in the context of a specific embodiment of horizontal axis laundry appliance 100, using the teachings disclosed herein it will be understood that horizontal axis laundry appliance 100 is provided by way of example only. Other laundry appliances having different configurations, different appearances, or different features may also be utilized with the present subject matter as well (e.g., vertical axis laundry appliances). For instance, aspects of the present subject matter may be applicable to dedicated dryers or drying appliances, as would be understood. Indeed, it should be appreciated that aspects of the present subject matter may further apply to other laundry appliances. In this regard, the same methods as systems and methods as described herein may be used to implement dry cycles for other appliances, as described in more detail below.

Referring again to FIG. 2, conditioning system 200 may include a return duct 220 that is mounted to tub 124 for circulating air within chamber 126 to facilitate a dry cycle (e.g., as part of a wash/dry or drying operation). For example, according to the illustrated exemplary embodiments, return duct 220 is fluid coupled to tub 124 proximate a top of tub 124. Return duct 220 receives heated air that has been heated or dehumidified by a conditioning system 200 and provides the heated air to laundry basket 120 via one or more holes defined by rear wall 206 or cylindrical wall 208 of laundry basket 120 (e.g., such as perforations 140).

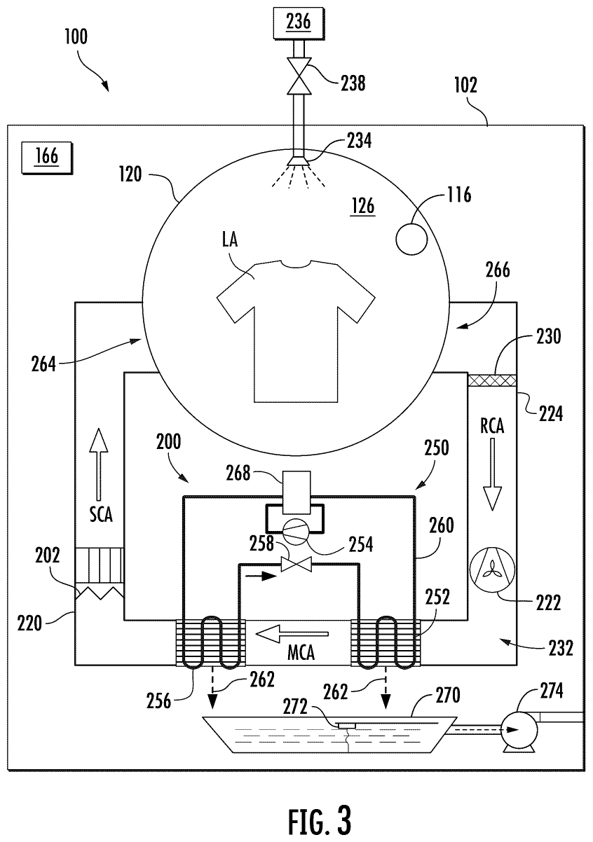

During a dry cycle, moisture laden, heated air is drawn from laundry basket 120 by an air handler, such as a blower fan 222, which may generate a negative air pressure within laundry basket 120. As the air passes from blower fan 222, it enters an intake duct 224 and then is passed into conditioning system 200. In some embodiments, the conditioning system 200 may have a heater 202 that includes or is provided as an electric heating element (e.g., a resistive heating element) or a gas-powered heating element (e.g., a gas burner), as would be understood. According to the illustrated exemplary embodiment, laundry appliance 100 is a heat pump laundry appliance and thus conditioning system 200 may be or include a heater including a heat pump having a sealed refrigerant circuit, as described in more detail below with reference to FIG. 3. Conditioning air (e.g., heated air with a lower moisture content than was received from laundry basket 120), may exit conditioning system 200 and returns to laundry basket 120 by a return duct 220. As air is heated and circulated through the chamber 126, the basket 120 may be rotated (e.g., as motivated by the motor 122), such as at a set tumble speed, to permit agitation (e.g., at non-plastering or sub-plaster speeds), as is understood. After the clothing articles have been dried (e.g., following completion of the dry cycle), the articles may be removed from the laundry basket 120 via opening 132.

In some embodiments, conditioning system 200 may be operated during a cooling cycle (e.g., as part of a drying operation or laundry operation, generally). During a cooling cycle, air is drawn from laundry basket 120 by blower fan 222, which may generate a negative air pressure within laundry basket 120. As the air passes from blower fan 222, it enters an intake duct 224 and then is passed into conditioning system 200. As will be described in greater detail below, conditioning system 200 may be configured to selectively cool (e.g., actively draw heat from) air from intake duct 224 and to basket 120. Conditioning air (e.g., cooled air), may exit conditioning system 200 and returns to laundry basket 120 by return duct 220. As air is cooled and circulated through the chamber 126, the basket 120 may be rotated (e.g., as motivated by the motor 122), such as at a set tumble speed, to permit agitation (e.g., at non-plastering or sub-plaster speeds), as is understood. After the clothing articles or air within laundry chamber 126 have/has been cooled (e.g., following completion of the cooling cycle), one or more further actions may be directed, such as a dry cycle described above.

As shown, laundry appliance 100 may further include one or more lint filters 230 (FIG. 3) to collect lint during operations (e.g., a drying operation or laundry operation, generally). The moisture laden air passes through intake duct 224 enclosing screen filter 230, which traps lint particles. More specifically, filter 230 may be placed into an air flow path 232 defined by laundry basket 120, conditioning system 200, intake duct 224, and return duct 220. Filter 230 may be positioned in the process air flow path 232 and may include a screen, mesh, other material to capture lint in the air flow 232. The location of lint filters in laundry appliance 100 as shown in FIG. 3 is provided by way of example only, and other locations may be used as well. According to exemplary embodiments, lint filter 230 is readily accessible by a user of the appliance. As such, lint filter 230 should be manually cleaned by removal of the filter, pulling or wiping away accumulated lint, and then replacing the filter 230 for subsequent drying or dry cycles.

According to optional embodiments, laundry appliance 100 may facilitate an ozone cleaning cycle (e.g., as part of a drying operation or laundry operation, generally). In this regard, laundry appliance 100 may offer the ozone cleaning cycle, during which ozone is injected into chamber 126 (e.g., to reduce or eliminate various microbes within chamber 126). Accordingly, as shown for example in FIG. 3, laundry appliance 100 may include an ozone nozzle 234 that is in fluid communication with an ozone generator 236 (e.g., mounted within cabinet) in order to direct ozone into chamber 126. Ozone generator 236 may be any suitable structure or assembly for producing selectively-producing gaseous ozone, such as a corona discharge ozone generator 236, ultraviolet light ozone generator 236, etc. When assembled, ozone generator 236 may be operably coupled to controller 166 and selectively activated to generate gaseous ozone (e.g., as directed by controller 166). Optionally, laundry appliance 100 may further include a control valve or gate 238 for selecting permitting the flow of ozone into chamber 126. It should be appreciated that control valve 238 may be positioned at any suitable location within cabinet 102.

FIG. 3 provides a schematic view of laundry appliance 100 and depicts conditioning system 200 in more detail. In the illustrated embodiments, laundry appliance 100 is a heat pump dryer appliance and thus conditioning system 200 includes a sealed system 250. Sealed system 250 includes various operational components, which can be encased or located within a machinery compartment of laundry appliance 100. In some embodiments, the operational components are operable to execute a vapor compression cycle for heating or cooling air passing through conditioning system 200 and to the chamber 126.

The operational components of sealed system 250 include a first heat exchanger (HE) coil (e.g., acting as an evaporator in a heating mode) 252, a compressor 254, a second HE coil (e.g., acting as a condenser in a heating mode) 256, and one or more expansion devices 258 connected in series along a refrigerant circuit or line 260. Refrigerant line 260 is charged with a working fluid, which in this example is a refrigerant.

In some embodiments, such as exemplary heat pump unit embodiments, the sealed system includes a reversible refrigerant valve 268. Reversible refrigerant valve 268 selectively directs compressed refrigerant from compressor 254 to either first HE coil 252 or second HE coil 256. For example, in a cooling mode, reversible refrigerant valve 268 110 is arranged or configured to direct compressed refrigerant from compressor 254 to first HE coil 252. Conversely, in a heating mode, reversible refrigerant valve 268 110 is arranged or configured to direct compressed refrigerant from compressor 254 to second HE coil 256.

Sealed system 250 depicted in FIG. 3 is provided by way of example only. Thus, it is within the scope of the present subject matter for other configurations of the sealed system to be used as well. As will be understood by those skilled in the art, sealed system 250 may include additional components (e.g., at least one additional heat exchanger, compressor, expansion device, etc.). For instance, sealed system 250 may include two first heat exchangers (e.g., to selectively operate as evaporators).

In performing a dry cycle, one or more laundry articles LA may be placed within the chamber 126 of laundry basket 120. For instance, following a wash cycle, articles may remain within the chamber 126.

Post-conditioning air SCA may be supplied to chamber 126 via return duct 220. The post-conditioning air SCA enters chamber 126 of laundry basket 120 via a tub inlet 264 defined by laundry basket 120 (e.g., the plurality of holes defined in rear wall 206 or cylindrical wall 208 of laundry basket 120 as shown in FIG. 2). In a cooling mode, post-conditioning air SCA may have a relatively low temperature (e.g., below 50° Fahrenheit) to reduce the temperature of laundry articles LA or chamber 126, generally. Conversely, in a heating mode, the post-conditioning air SCA provided to chamber 126 may have a relatively high temperature (e.g., above 120° Fahrenheit), and may cause moisture within laundry articles LA to evaporate.

Subsequently, air within chamber 126 exits chamber 126 as pre-conditioning air RCA. The pre-conditioning air RCA exits chamber 126, such as through a tub outlet 266 defined by laundry basket 120 and flows into intake duct 224. After exiting chamber 126 of laundry basket 120, the pre-conditioning air RCA may flow downstream to conditioning system 200. Blower fan 222 moves the pre-conditioning air RCA, as well as the air more generally, through a process air flow path 232 defined by laundry basket 120, conditioning system 200, intake duct 224, and return duct 220. Thus, generally, blower fan 222 is operable to move air through or along the process air flow path 232. The duct system includes all ducts that provide fluid communication (e.g., airflow communication) between tub outlet 266 and conditioning system 200 and between conditioning system 200 and tub inlet 264. Although blower fan 222 is shown positioned between laundry basket 120 and conditioning system 200 along intake duct 224, it will be appreciated that blower fan 222 can be positioned in other suitable positions or locations along the duct system.

As further depicted in FIG. 3, the pre-conditioning air RCA flows into or across first HE coil 252 of the conditioning system 200. As the pre-conditioning air RCA passes across first HE coil 252, the temperature of the air is either increased (e.g., in a cooling mode) or reduced (e.g., in a heating mode) through heat exchange with refrigerant that is within, for example, coils or tubing of first HE coil 252. The heat exchange may condense (e.g., in a cooling mode) or vaporize (e.g., in a heating mode) refrigerant within first HE coil 252.

As shown in FIG. 3, mid-conditioning air MCA (cool or hot relative to pre-conditioning air RCA) flowing downstream of first HE coil 252 is subsequently caused to flow across second HE coil 256 (e.g., across coils or tubing thereof), which vaporizes (e.g., in a cooling mode) or condenses (e.g., in a heating mode) refrigerant within second HE coil 256. As a result, heat energy is either drawn from or transferred to the mid-conditioning air MCA at the second HE coil 256, thereby reducing or elevating its temperature and providing cool or warm post-conditioning air SCA for resupply to laundry basket 120 of laundry appliance 100. The post-conditioning air SCA passes over and around laundry articles LA within the chamber 126 of the laundry basket 120, such that pre-conditioning air RCA is generated, as mentioned above.

It is noted that air passing over first or second HE coil 252 or 256 may have moisture that is condensed on top of or against the first HE coil 252 (e.g., in a heating mode) or the second HE coil 256 (e.g., in a cooling mode). In optional embodiments, moisture in the air is condensed and such condensate water may be drained from conditioning system 200 (e.g., using a drain line 262, which is also depicted in FIG. 3).

In optional embodiments, a condenser tank or a condensate collection tank 270 is in fluid communication with conditioning system 200 (e.g., via drain line 262). Collection tank 270 is operable to receive condensate water from the process air flowing through conditioning system 200, and more particularly, condensate water from first HE coil 252. A sensor 272 may be operable to detect when water within collection tank 270 has reached a predetermined level. Sensor 272 can be any suitable type of sensor, such as a float switch as shown in FIG. 3. Sensor 272 can be communicatively coupled with controller 166 (e.g., via a suitable wired or wireless communication link). A drain pump 274 is in fluid communication with collection tank 270. Drain pump 274 is operable to remove a volume of water from collection tank 270 and, for example, discharge the collected condensate to an external drain. In some embodiments, drain pump 274 can remove a known or predetermined volume of water from collection tank 270. Drain pump 274 can remove the condensate water from collection tank 270 and can move or drain the condensate water downstream (e.g., to a gray water collection system). Particularly, in some embodiments, controller 166 is configured to receive, from sensor 272, an input indicating that water within the collection tank has reached the predetermined level. In response to the input indicating that water within collection tank 270 has reached the predetermined level, controller 166 can cause drain pump 274 to remove the predetermined volume of water from collection tank 270.

With respect to sealed system 250, compressor 254 pressurizes refrigerant (i.e., increases the pressure of the refrigerant) passing therethrough and generally motivates refrigerant through the sealed refrigerant circuit or refrigerant line 260 of conditioning system 200. Compressor 254 may be communicatively coupled with controller 166 (communication lines not shown in FIG. 3). Refrigerant is supplied from either the second HE coil 256 (e.g., in a cooling mode) or the first HE coil 252 (e.g., in a heating mode) to compressor 254 in a low pressure gas phase. The pressurization of the refrigerant within compressor 254 increases the temperature of the refrigerant. The compressed refrigerant is fed from compressor 254 to first or second HE coil 252 or 256 through refrigerant line 260. As the relatively mid-conditioning air MCA flows, the refrigerant is cooled and its temperature is lowered as heat is transferred to the air for supply to chamber 126 of laundry basket 120.

Upon exiting first or second HE coil 256, the refrigerant is fed through refrigerant line 260 to expansion device 258. Although only one expansion device 258 is shown, such is by way of example only. It is understood that multiple such devices may be used. In some embodiments, expansion device 258 is an electronic expansion valve, although a thermal expansion valve capillary tube or any other suitable expansion device can be used. Generally, expansion device 258 lowers the pressure of the refrigerant and controls the amount of refrigerant that is allowed to enter the second HE coil 256 or first HE coil 252. Importantly, the flow of liquid refrigerant into second HE coil 256 or first HE coil 252 is limited by expansion device 258 in order to keep the pressure low and allow expansion of the refrigerant back into the gas phase in second HE coil 256 or first HE coil 252. The evaporation of the refrigerant in second HE coil 256 or first HE coil 252 converts the refrigerant from its liquid-dominated phase to a gas phase while cooling air across the opposite HE coil (e.g., first HE coil 252 or second HE coil 256). The process is repeated as air is circulated along process air flow path 232 while the refrigerant is cycled through sealed system 250, as described above.

In the case of a tumble dry cycle, the heater (e.g., sealed system 250) remains inactive such that heat is not actively generated or, alternatively, the heater may be directed to a relatively low heat setting (i.e., a first heat setting that is lower in power, voltage, duty cycle, or temperature than a second heat setting of the dry cycle). For instance, the compressor 254 may be directed to a reduced state. Optionally, compressor 254 may be held inactive to restrict the flow of refrigerant through sealed system 250. Nonetheless, air may be cycled through chamber 126 along the same path as air circulated during a dry cycle (e.g., as described above).

As shown in FIGS. 2 and 3 one or more measurement devices 180 may be provided in the washing machine appliance 100 for measuring one or more conditions within the laundry chamber 126, such as during a dry cycle of a drying operation. Measurement devices 180 may measure a variety of suitable variables such as temperature (e.g., as or including a thermistor or thermocouple) or ozone levels (e.g., as or including an electrochemical ozone detector).

Now that the construction of laundry appliance 100 and the configuration of controller 166 according to exemplary embodiments have been presented, an exemplary method 400 of operating a laundry appliance will be described. Although the discussion below refers to the exemplary method 400 of operating laundry appliance 100, one skilled in the art will appreciate that the exemplary method 400 is applicable to the operation of a variety of other laundry appliances. In exemplary embodiments, the various method steps as disclosed herein may be performed by controller 166 (e.g., as a drying operation) or a separate, dedicated controller.

It is noted that the order of steps within method 400 is for illustrative purposes. All may be adopted or characterized as being fulfilled in a common operation. Except as otherwise indicated, one or more steps in the below method 400 may be changed, rearranged, performed in a different order, or otherwise modified without deviating from the scope of the present disclosure.

Advantageously, embodiments described herein may provide a laundry appliance or method facilitating ozone-sanitization (e.g., reliably, efficiently, quickly, or while otherwise ensuring a user is not exposed to excessive ozone levels).

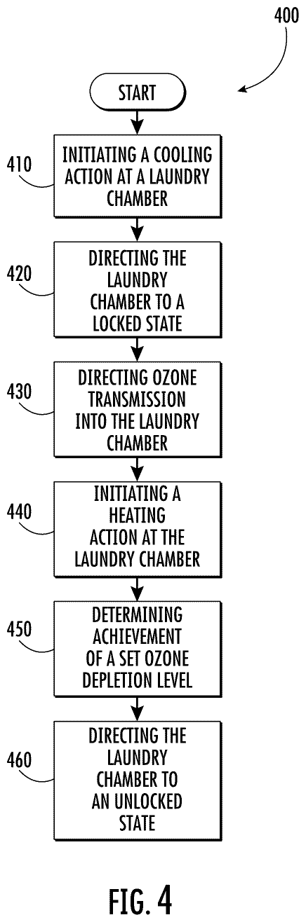

Referring now to FIG. 4, at 410, the method 400 includes

At 410, the method 400 includes initiating a cooling action at a laundry chamber to reduce a temperature within the laundry chamber. For instance, 410 may be provided with or as part of a drying operation for articles within the laundry basket. In particular, the drying operation may include a selection or option for ozone cleaning. Optionally, the drying operation is part of a wash/dry operation includes a discrete wash cycle and dry cycle (e.g., as described above). In other words, the wash/dry operation may include a first cycle in which the articles are wetted and washed (e.g., as described above) and a separate second cycle that follows the first cycle and in which the wetted/washed articles are actively heated or dried (e.g., as described above). Thus, 410 may prompt the wash cycle or dry cycle and cause the same to be executed. The cooling action may occur prior to one or both of the wash cycle and the dry cycle.

As would be understood, initiation of the drying operation may be prompted by any suitable input from a user. For instance, a user may engage (e.g., press, turn, toggle, touch, etc.) the user interface of the laundry appliance to select the wash/dry operation (e.g., including attributes thereof) or otherwise register an intent for the drying operation to begin.

The cooling action of 410 may generally include reducing the temperature of articles or air within the laundry chamber. For instance, a sealed refrigerant system may be directed to cool air entering the laundry chamber through one or more air ducts (e.g., as described above). Thus, air may be motivated across a heat exchange coil acting as an evaporator (e.g., without subsequent heating) and into the laundry chamber. As described above, the sealed refrigerant system may be provided as a reversible heat pump system. In turn, a reversible refrigerant valve may be directed or moved to a cooling mode position. Optionally, the cooling action may include rotating the laundry basket (e.g., oscillating back-and-forth or traveling greater than 360° through full rotation). In turn, articles within the laundry chamber may be agitated or tumbled as relatively cold air is blown into the laundry chamber, thereby reducing the temperature within laundry chamber.

At 420, the method 400 includes directing the laundry chamber to a locked state. Thus, the door to the laundry chamber may be locked (e.g., by actuation of the latch assembly, as would be understood). The locking of the laundry chamber may generally coincide with or follow 410 (e.g., with or in response to 410). Moreover, the locked state may be directed to continue during 430, as described below.

At 430, the method 400 includes directing ozone transmission into the laundry chamber following 410. Specifically, the ozone generator in fluid communication with the laundry chamber may be activated or otherwise instructed to generate ozone, which is then transferred to transmitted to the laundry chamber (e.g., according to a suitable set transmission rate, pattern, or schedule—as would be understood). If present, a control valve may be opened to permit the flow of ozone to the laundry chamber. For the duration of ozone transmission, the laundry chamber may remain in the locked state. Moreover, the laundry appliance may be sealed (e.g., due in part to sealed engagement between the door and gasket or baffle), as would be understood to restrict the escapement of ozone from the laundry chamber to the surrounding room in which the laundry appliance is disposed. In some embodiments, the cooling action of 410 continues for all or at least a portion of ozone transmission. Thus, 430 may be simultaneous to at least a portion of 410.

Following completion or upon reaching the end of 430, it is understood that the ozone generator may be directed to an inactive state. Moreover, if present, the control valve may be closed. Thus, transmission of ozone may be stopped or otherwise ended.

At 440, the method 400 includes initiating a heating action at the laundry chamber after 430 (e.g., following completion or ending of ozone transmission). Specifically, 440 may increase the temperature (e.g., of articles or air) within the laundry chamber following directing ozone transmission. Optionally, 440 may include activating an electric heating element or heater in thermal communication with the laundry chamber (e.g., on or within an air duct thereto, as described above). Additionally or alternatively, 440 may include directing the sealed refrigerant system to heat air entering the laundry chamber through one or more air ducts (e.g., as described above). Thus, air may be motivated across a heat exchange coil acting as a condenser (e.g., without subsequent cooling) and into the laundry chamber. As described above, the sealed refrigerant system may be provided as a reversible heat pump system. In turn, the reversible refrigerant valve may be directed or moved to a heating mode position. Optionally, the heating action may include or be provided as part of a dry cycle (e.g., as described above).

In some embodiments, the method 400 includes determining a set ozone level is reached during ozone transmission, such as by a direct measurement (e.g., one or more sensor readings of ozone levels within laundry chamber) or an indirect proxy (e.g., reaching a predetermined transmission time limit for ozone transmission). As an example, a timer sequence may be initiated in tandem with the start of 430, upon reaching a predetermined transmission time limit (e.g., based on empirical testing of a representative unit) for duration of the ozone transmission, it may be determined that the set ozone level is reached. In response to determining the set ozone level is reached, the ozone transmission may be halted or the heating action may otherwise be initiated.

At 450, the method 400 includes determining achievement of a set ozone depletion level within the laundry chamber following 440. In particular, it may be determined that the ozone levels within the laundry chamber have been reduced to below a maximum threshold (e.g., the set ozone depletion level). The determination of 450 may be made using a direct measurement (e.g., one or more sensor readings of ozone levels or temperature within laundry chamber) or an indirect proxy (e.g., reaching a predetermined heating time limit for the heating action). As an example, a timer sequence may be initiated in tandem with the start of 440, upon reaching a predetermined heating time limit (e.g., based on empirical testing of a representative unit) for duration of the heating action, it may be determined that the set ozone depletion level is reached.

At 460, the method 400 includes directing the laundry chamber to an unlocked state (e.g., following or in response to 450, such upon completion of a dry cycle, as would be understood). Thus, the door to the laundry chamber may be unlocked (e.g., by withdrawal of the latch assembly, as would be understood). The unlocking of the laundry chamber may generally coincide with or follow 450 (e.g., with or in response to 450). Thus, following reduction of the ozone within the laundry chamber, the door may be unlocked to permit a user to remove articles within the chamber.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A laundry appliance comprising:
a cabinet;
a tub positioned within the cabinet, the tub defining a tub outlet and a tub inlet;
a laundry basket rotatably mounted within the tub, the laundry basket defining a laundry chamber for receipt of articles for washing or drying;
a conditioning system comprising a heat exchanger disposed within the cabinet in thermal communication with the laundry chamber to adjust a temperature therein; and
a controller operably coupled to the conditioning system and configured to initiate a laundry operation, the laundry operation comprising
initiating a cooling action at the heat exchanger to reduce a temperature within the laundry chamber,
directing ozone transmission into the laundry chamber following initiating the cooling action,
initiating a heating action at the laundry chamber to increase the temperature within the laundry chamber following directing ozone transmission, and
determining achievement of a set ozone depletion level within the laundry chamber following initiating the heating action.

2. The laundry appliance of claim 1, wherein the conditioning system comprises a sealed refrigerant system in thermal communication with the laundry chamber.

3. The laundry appliance of claim 2, wherein the sealed refrigerant system comprises a compressor, a first heat exchange coil, a second heat exchange coil, and a reversible refrigerant valve disposed along a refrigerant line, wherein the reversible refrigerant valve is movable between a cooling mode position directing refrigerant flow from the compressor to the first heat exchange coil and a heating mode position directing refrigerant flow from the compressor to the second heat exchange coil.

4. The laundry appliance of claim 3, wherein initiating the cooling action comprises directing the reversible refrigerant valve to the cooling mode position.

5. The laundry appliance of claim 3, wherein initiating the cooling action comprises directing the reversible refrigerant valve to the heating mode position.

6. The laundry appliance of claim 1, wherein determining ozone depletion comprises determining a set heating time is reached for the heating action.

7. The laundry appliance of claim 1, wherein directing ozone transmission is simultaneous to at least a portion of the cooling action.

8. The laundry appliance of claim 1, wherein the laundry operation further comprises determining a set ozone level is reached during ozone transmission, wherein initiating the heating action is in response to determining the set ozone level is reached.

9. The laundry appliance of claim 1, wherein the laundry operation further comprises directing the laundry chamber to a locked state for ozone transmission.

10. The laundry appliance of claim 9, wherein the laundry operation further comprises directing the laundry chamber to an unlocked state in response to determining achievement of the set ozone depletion level.

11. A method of operating a dryer appliance comprising a cabinet, a laundry basket rotatably mounted within the cabinet, and a conditioning system comprising a heat exchanger in thermal communication with the laundry basket, the method comprising:
initiating a cooling action at the heat exchanger to reduce a temperature within a laundry chamber;
directing ozone transmission into the laundry chamber following initiating the cooling action;
initiating a heating action at the laundry chamber to increase the temperature within the laundry chamber following directing ozone transmission; and
determining achievement of a set ozone depletion level within the laundry chamber following initiating the heating action.

12. The method of claim 11, wherein the conditioning system comprises a sealed refrigerant system in thermal communication with the laundry chamber.

13. The method of claim 12, wherein the sealed refrigerant system comprises a compressor, a first heat exchange coil, a second heat exchange coil, and a reversible refrigerant valve disposed along a refrigerant line, wherein the reversible refrigerant valve is movable between a cooling mode position directing refrigerant flow from the compressor to the first heat exchange coil and a heating mode position directing refrigerant flow from the compressor to the second heat exchange coil.

14. The method of claim 13, wherein initiating the cooling action comprises directing the reversible refrigerant valve to the cooling mode position.

15. The method of claim 13, wherein initiating the cooling action comprises directing the reversible refrigerant valve to the heating mode position.

16. The method of claim 11, wherein determining ozone depletion comprises determining a set heating time is reached for the heating action.

17. The method of claim 11, wherein directing ozone transmission is simultaneous to at least a portion of the cooling action.

18. The method of claim 11, further comprising determining a set ozone level is reached during ozone transmission, wherein initiating the heating action is in response to determining the set ozone level is reached.

19. The method of claim 11, further comprising directing the laundry chamber to a locked state for ozone transmission.

20. The method of claim 19, further comprising directing the laundry chamber to an unlocked state in response to determining achievement of the set ozone depletion level.
